(19) United States
(12) Patent Application Publication
(10) Pub. No.: US 2011/0224299 A1
(43) Pub. Date: Sep. 15, 2011

(54) Stabilized Wax Composition and Uses Thereof

(75) Inventors: William E. McNamara, Chester, NY (US); Mark S. Garrison, Suffern, NY (US); Thi N. Do, West Orange, NJ (US)

(73) Assignee: Avon Products, Inc., Suffern, NY (US)

(21) Appl. No.: 12/839,692

(22) Filed: Jul. 20, 2010

Related U.S. Application Data
(60) Provisional application No. 61/239,500, filed on Sep. 3, 2009.

(51) Int. Cl. A61K 8/92; A61Q 19/00

(52) U.S. Cl. 514/552; 514/762

(57) ABSTRACT

Compositions comprising high amounts of low melting point waxes are provided in self-supporting solid or semi-solid form. The low melting point waxes are stabilized in the compositions by the presence of small amounts of higher melting wax. The compositions may provide a cooling sensation when applied to the skin.

### RELATED APPLICATIONS

This application claims priority to U.S. Provisional Patent Application Ser. No. 61/239,500, filed Sep. 3, 2009, the contents of which are hereby incorporated by reference in their entirety.

### FIELD OF INVENTION

The present invention relates generally to compositions for convenient topical application of low melting point waxes to human skin, and in particular, the lips.

### BACKGROUND OF THE INVENTION

Low melt materials, such as waxes having a melting point below about 30° C., for use in cosmetics and other applications present formulation challenges because these materials are typically liquid or very soft semi-solids at room temperature. Conventionally, to achieve reasonable stability of solid formulations at room temperature, only relatively small proportions of low melt materials could be employed. Often, a large proportion of high melt wax is included to stabilize small amounts of low melt wax. However, the may limit the cosmetic effectiveness of the low melt components and may produce a waxy feel and diminished payoff when applied to the skin. In purer states, in contrast, low melt materials can produce cooling sensations on the skin, as well as a softer, more luxurious feel, but effective approaches to produce cooling cosmetic compositions based on low melt materials have been limited.

Some approaches have looked to the use of hollow particles to contain low melt materials, such as fatty acid esters, in low relative amounts. For example, hollow particles, such as those sold under trade-names ORGASOL™ and EXPANCEL™, have been used to contain fatty acid monoesters in low amounts. A related approach involves the use of microcapsules. For example, heat-stabilizing microcapsules have been described that contain crystalline compounds in leak-tight envelopes, where the crystalline compounds act as heat buffers, absorbing heat and so buffering the temperature of the immediate environment.

Other approaches involve the use of volatile fluids or emulsions that exclude oil-soluble components to help stabilize the low melt materials. Volatile silicone, for example, has been described as part of a hybrid silicone matrix for holding hydrophobic ingredients; as have emulsions that stabilize an oil-soluble coolant in its crystalline state by using a polymeric emulsifier and only water-soluble materials.

Nonetheless, there remains a need in the cosmetic arts for improved stable cosmetic compositions comprising low melt materials, particularly where such compositions could be used to provide sensory cooling effects. It is therefore an object of the invention to provide compositions and methods addressing these and other needs.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, it has surprisingly been found that cosmetic compositions comprising a first waxy component which melts at relatively low temperatures and which, by itself does not form a self-supporting solid at room temperature, can be stabilized by the presence of modest amounts of a second wax component melting at relatively high temperatures, such that the combination of wax components may advantageously exist as a self-supporting solid, for example, a stick, at room temperature (˜25° C.), yet readily transfer to the skin, with a high payoff, at body temperature (˜35° C.). The compositions are intended for application to human skin and provide a convenience and elegant product form for delivering low melt waxes to the skin. The compositions ideally provide a perceptible cooling sensation when applied to the skin due to the endothermic melting of the low melt wax component.

The compositions of the invention are typically in the form of self-supporting solids at room temperature and are characterized by high loads of low melt wax and will typically contain low melt waxes in an amount of at least about 25% by weight of the composition, although significantly higher loadings are possible without sacrificing the physical form of the product. For example, the compositions may comprise low melt waxes up to about 90% by weight or even higher and still exist as self-supporting solids at room temperature, whereas the low melting wax component, alone, is typically a liquid or very soft semi-solid at room temperature. The low melting point wax component will typically have a melting temperature below about 30° C.

Preferred waxes for the first wax component are characterized by a difference between onset and peak melting temperatures of less than about 12° C., and more preferably less than about 7° C., as measured by differential scanning calorimetry (DSC), such that a perceptible cooling sensation is detected when the first wax component melts on contact with human skin. The onset temperature, also known as the onset point, is the temperature at which the first change is observed during the melt, and represents the initiation of the phase change from solid to liquid. The peak temperature, also known as the liquefaction point, is the temperature at which the material becomes completely liquid and no more solid is left, and corresponds to the high temperature record in the melting temperature range. Without wishing to be bound by any particular theory, it is believed that the narrow range of melting temperatures of the preferred low melt waxes causes heat to be absorbed by the compositions in a shorter amount of time than would be the case where the melting range is broader and thus the perception of cooling on the skin is more pronounced because the heat transfer flux is increased. Octadecane (in particular, n-octadecane) and stearyl heptanoate have been found to be suitable low melt waxes for use according to the invention and excellent cooling results have been obtained with each.

The high melting point wax component will typically be present in amounts below about 40% by weight and often will be a minor component in comparison to the amount of low melt wax component. The high melt wax component will usually comprise from about 5% to about 30% by weight of the composition, and often will comprise from about 10% to about 20% by weight of the composition. What is important is that the amount of high melt wax is adequate to stabilize the low melt wax such that the combination exists as a self-supporting solid or semi-solid at room temperature. The identity of the high melt wax component is not particularly limited but should ideally have a melting temperature above about 50° C. In various embodiments, the melting temperature of the second wax component will be above about 60° C., above about 70° C., or even above about 80° C. The high melt wax may be selected, for example, from animal waxes, insect waxes, vegetable waxes, mineral waxes, petroleum waxes, and synthetic waxes, including, without limitation carnauba, paraffin, candelilla, castor, beeswax, microcrystalline, ceresin, ozokerite, polyethylene wax, polyalky acrylate, alkyl silicone, and combinations thereof. Of course, the high melting point wax component includes combinations of high melting point waxes. Paraffin wax has been found suitable for use as the high melting temperature wax component.

Because the preferred compositions provide a cooling sensation on application to the skin, products such as lip balms, sunburn treatment compositions, and compositions for treating sore muscles may particularly benefit from the inventive compositions. As collateral benefit, the low melting point waxes may provide a desirable moisturizing effect to the skin, including for example, the lips and other areas of skin susceptible to excessive dryness, such as the area under the eyes. Of the many other contemplated uses, a facial cleanser may be mentioned, and in particular a water-free facial cleanser.

These and other aspects of the invention will be better understood by reference to the following detailed description, drawings, and appended claims.

### BRIEF DESCRIPTION OF THE DRAWINGS

FIG. 1 is a DSC thermogram illustrating the melting characteristics of isostearyl hydrostearate, with heat flow (in mW) plotted against temperature (in ° C.).

FIG. 2 is a DSC thermogram illustrating the melting characteristics of glycerol tridecanoate, with heat flow (in mW) plotted against temperature (in ° C.).

FIG. 3 is a DSC thermogram illustrating the melting characteristics of 89% pure stearyl heptanoate, with heat flow (in mW) plotted against temperature (in ° C.).

FIG. 4 is a DSC thermogram illustrating the melting characteristics of 99% pure stearyl heptanoate, with heat flow (in mW) plotted against temperature (in ° C.).

FIG. 5 is a DSC thermogram illustrating the melting characteristics of a 90% stearyl heptanoate composition, with heat flow (in mW) plotted against temperature (in ° C.).

FIG. 6 is a DSC thermogram illustrating the melting characteristics of an 80% stearyl heptanoate composition, with heat flow (in mW) plotted against temperature (in ° C.).

FIG. 7 is a DSC thermogram illustrating the melting characteristics of octadecane, with heat flow (in mW) plotted against temperature (in ° C.).

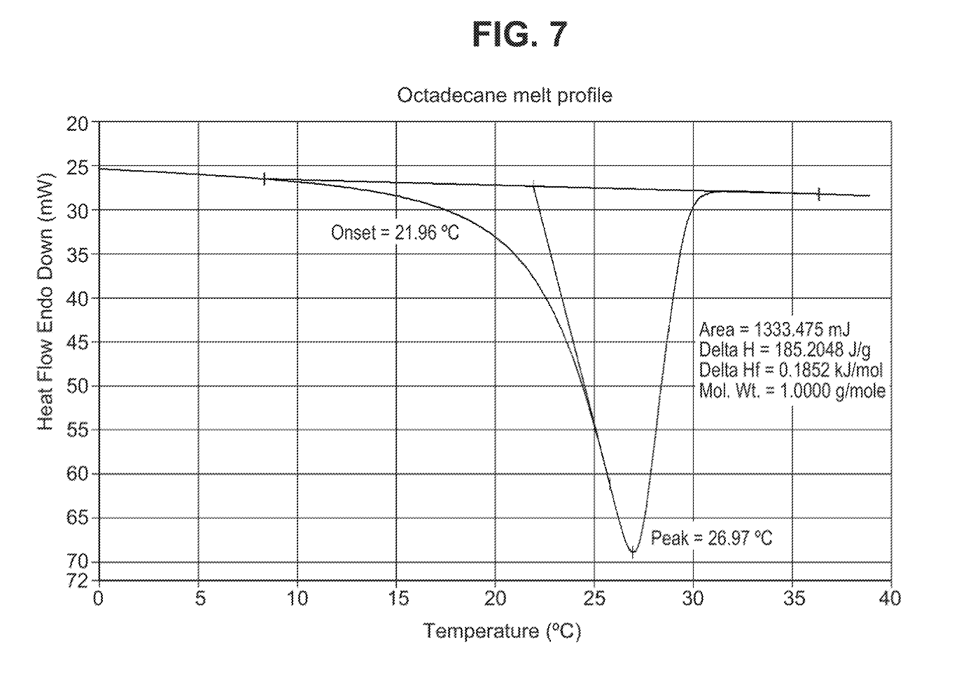

### DETAILED DESCRIPTION

The composition of the invention will comprise a preferably homogenous mixture of a low melting point wax component and a high melting point wax component in an amount sufficient to stabilize the composition as a self-supporting solid or semi-solid at room temperature.

“Low melt materials” refers to materials, such as waxes, having a melting point close to but below human skin temperature, so that the material only melts upon application to the skin. Preferably, the melting point is between about 15° C. and about 40° C., more preferably between about 25° C. and about 35° C., and most preferably just slightly below human skin temperature (about 35° C.). Low melt waxes described herein can absorb heat (e.g., from the skin) to undergo an endothermic phase transition from solid to liquid, a process that can be characterized by differential scanning calorimetry (DSC). Phases, such as the solid and liquid phases, can be described as a set of states of a physical system that have relatively uniform chemical composition and physical properties and may be characterized by density, crystal structure and index of refraction. Phase transitions are reversible, and can take place repeatedly.

Preferred low melt waxes also have distinct melting characteristics. While pure crystalline substances have a sharp melting point, materials such as waxes display a melting temperature range, with an onset temperature and a peak temperature defining the ends of the range. The onset temperature, also known as the onset point, is the temperature at which the first change is observed during the melt, and represents the initiation of the phase change from solid to liquid. The peak temperature, also known as the liquefaction point, is the temperature at which the material becomes completely liquid and no more solid is left, and corresponds to the high temperature record in the melting temperature range. In preferred embodiments, low melt waxes have a difference between onset temperature and a peak temperature of less than about 12° C., and preferably less than about 11° C., less than about 10° C., less than about 9° C., less than about 8° C., less than about 7° C., less than about 6° C., and may even be less than about 5° C., or less than about 4° C. A preferred range is from about 3° C. to about 10° C., a more preferred range is from about 3° C. to about 7° C., and more preferred still is the range from about 3° C. to about 6° C.

Another melt characteristic involves the enthalpy of fusion. Enthalpy of fusion (ΔH) refers to the amount of thermal energy which is absorbed or evolved for a unit mass (1 mole) of a material to change states from a solid to a liquid or vice versa. It also is known as the latent heat of fusion and the temperature at which it occurs corresponds to the melting point of the material. A material's melting point, enthalpy of fusion, and melting temperature range can vary depending on the purity of the material. For example, such parameters can be used in purity determinations.

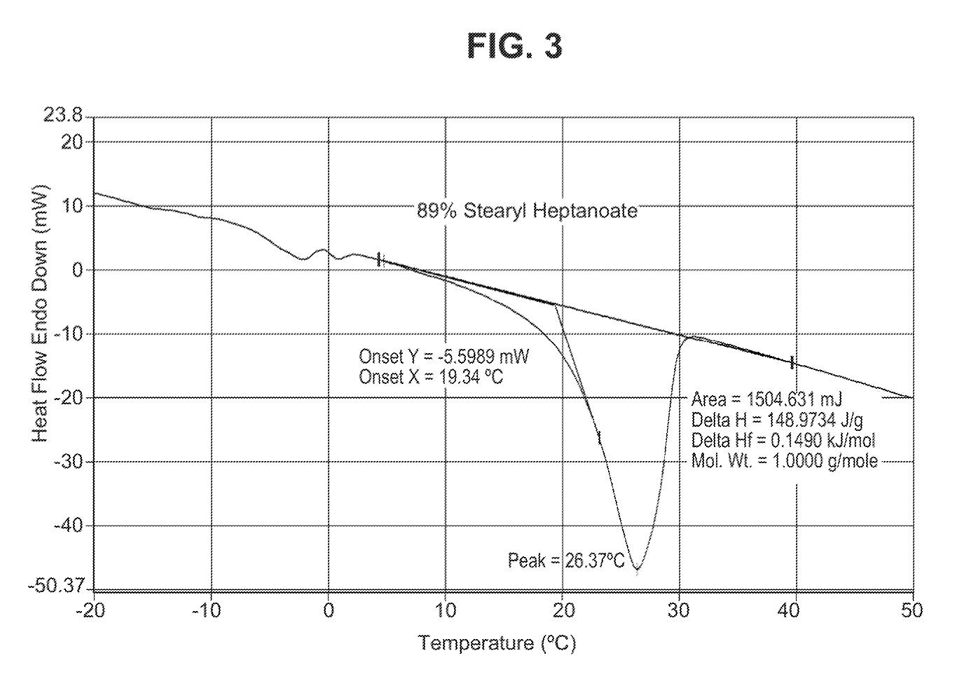

In some embodiments, the low melt waxes employed comprise fatty acid esters, more preferably fatty acid esters with a melting point of about 10° C. to about 35° C., or from about 20° C. to about 30° C. and preferably having a difference between onset and peak melting temperatures as specified herein. One such material is the fatty acid ester stearyl heptanoate, and melting temperature curves for various compositions comprising this ester are described herein in Examples 3-6 and FIGS. 3-6. Stearyl heptanoate is the ester of stearyl alcohol and heptanoic acid, and is useful in cosmetic compositions, for example, as an emollient.

In certain preferred embodiments, the low melt waxes employed comprise higher chain alkanes, including for example, n-pentadecane (MP ˜8-10° C.), n-hexadecane (MP 18° C.), n-heptadecane (MP ˜22° C.), n-octadecane (MP ˜28-30° C.), n-nonadecane (MP ˜32° C.), and n-eicosane (MP ˜37° C.) and all branched isomers of higher alkanes having melting temperatures in the range of about 5° C. to about 40° C., preferably about 10° C. to about 35° C., and preferably having a difference between onset and peak melting temperatures of less than about 12° C., more preferably less than about 7° C. A particularly preferred higher chain alkane is octadecane, and more particularly n-octadecane. n-Octadecane, as well as derivatives thereof, are particular preferred as the low melt wax in certain embodiments. Additional mention may be made of n-hexadecane, n-heptadecane, and n-nonadecane for use according to the present invention.

It is within the skill in the art to select suitable low melt materials that can be used in the practice of the instant invention. For example, other fatty acid esters, as well as paraffin waxes having suitable melting temperatures, silicone waxes and derivatives thereof, including eutectic mixtures falling into the aforementioned ranges, can be used. Table 1 below provides a number of suitable low melt waxes, including n-octadecane and stearyl heptanoate, along with their trade names and chemical formulae, as well as their melting points (MP) and enthalpies of fusion (Delta H) at a stated purity.

TABLE 1

| Low melt wax | formula | MP ° C. | ΔH J/g | % purity |
|---|---|---|---|---|
| stearyl heptanoate¹ | C₂₅H₅₀O₂ | 27 | 164 | 100 |
| fatty acid ester² | CₙH₂ₙO₂ | 27 | 198 | 100 |
| fatty acid ester³ | CₙH₂ₙO₂ | 32 | 224 | 100 |
| methyl palmitate | C₁₇H₃₄O₂ | 32-34 | | 98 |
| ethyl pamitate | C₁₈H₃₆O₂ | 26-27 | | 99 |
| methyl heptadecanoate | C₁₈H₃₆O₂ | 31-32 | | 99 |
| paraffin⁴ | CₙH₂ₙ₊₂ | 35 | 218 | 99 |
| n-Hexadecane | C₁₆H₃₄ | 18 | | 95 |
| n-Heptadecane | C₁₇H₃₆ | 22 | | 95 |
| n-Octadecane | C₁₈H₃₈ | 28 | | 90+ |
| n-Nonadecane | C₁₉H₄₀ | 32 | | 90+ |
| n-Eicosane | C₁₂H₄₂ | 37 | | 90+ |

¹Crodamol W;
²Astorphase 27;
³Astorphase 32;
⁴HA 18

Other potential examples include cocoa butter (melting point 31-35° C.) and illipe butter (melting point 34-38° C.). Additional examples of low melt waxes, along with methods for making same, are described in U.S. Pat. No. 7,226,502, the disclosure of which is hereby incorporated by reference.

Low melt materials used in the various embodiments of the instant invention are generally stabilized by combination with a high melt wax1 component. “Stabilized,” and related terms such as “stable,” “stability,” “stabilized,” “heat-stabilized,” and the like, refer to the ability of compositions comprising low melt materials to remain as self-supporting solids or semi-solids at room temperature and elevated temperatures (e.g., ˜40° C., ˜45° C., ˜50° C., etc.) while maintaining their integrity; and/or the ability of product forms comprising the compositions to regain their form (size, shape, texture, etc) and desired properties after exposure to elevated temperatures. “Stability” also refers to having a hardness sufficient for forming self-supporting forms, such as sticks and the like. “Elevated temperatures” refers herein to temperatures of about 40° C., about 45° C., about 47° C., about 49° C., about 50° C., or about 53° C. Preferably, stability can be maintained at elevated temperatures without material softening so as to not be self-supporting for extended periods of time, such as, e.g., a few minutes, an hour, preferably a few hours, more preferably a day, a few days, and still more preferably a week, a few weeks, a few months, or more, including for an indefinite period of time.

In some embodiments, the compositions comprise at least one low melt wax and a small proportion of at least one high melt wax, by which is meant below 40% by weight. A high melt wax refers to one having a higher melting point than that of a low melt material, typically at least 10° C. higher, at least 15° C. higher, at least 20° C. higher, or at least 25° C. higher, and preferably a melting point that is at least about 50° C. More preferably the melting point of the high melt wax is at least about 55° C., at least about 60° C., at least about 65° C., at least about 70° C. at least about 75° C., at least about 80° C., at least about 85° C., at least about 90° C., at least about 95° C., at least about 100° C., or higher. High melt waxes also typically, though not necessarily, have a softening point between about 50° C. and 90° C. One of skill in the art will recognize that high melt waxes are generally hydrophobic, and not considered water-soluble materials.

High melt waxes include, without limitation, many traditional waxes that are derived, for example, from animals, insects, vegetables, minerals, or petroleum, as well as synthetic waxes, fisher tropsch waxes, and mixtures of any of the foregoing waxes. In some embodiments, for example, the high melt wax is selected from carnauba, paraffin wax, candelilla, castor, beeswax, microcrystalline wax, ceresin, ozokerite, polyethylene wax, low MW polyalky acrylate, and silicone waxes, such as alkyl silicones, or any combinations thereof. Preferred embodiments employ paraffin waxes, silicone waxes, such as alkyl dimethicone and alkyl methicone waxes, having a melting point over about 50° C. and a softening point between about 50° C. and about 90° C. Table 2 below provides a number of suitable high melt waxes, along with their corresponding melting points (MP).

TABLE 2

| high melt wax | ~MP ° C. |
|---|---|
| acrawax | 140 |
| microcrystalline petroleum wax | 99 |
| linear polyethylene wax | 95 |
| stearone | 89 |
| castor wax | 86 |
| montan wax | 82-95 |
| lignite wax | 82-95 |
| ouricouri wax | 81-84 |
| carnauba wax | 78-85 |
| rice bran wax | 77-86 |
| shellac wax | 74-78 |
| esparto wax | 73 |
| ozokerite wax | 72 |
| jojoba wax | 70 |
| candelilla wax | 68-73 |
| ceresin wax | 67-71 |
| beeswax | 62-64 |
| castor wax | 60 |
| sugarcane wax | 60 |
| stearyl alcohol | 59 |
| hard tallow | 57-60 |
| cetyl alcohol | 56 |
| petrolatum | 54 |
| glyceryl monostearate | 54-56 |
| Japan wax | 53 |
| lanolin alcohol | 45-60 |

Without wishing to be bound by theory, it is believed that the combination of high melt wax with low melt wax allows for stabilization of the low melt material, while the use of a small amount of the high melt material compared to the low melt material allows for optimization of certain desirable properties of the low melt material. That is, using the proportions of low melt to high melt wax taught herein can produce synergistic combinations, such that only a small proportion of high melt wax, e.g., 5-30% by weight, 7.5-25% by weight, or 10-20% by weight based on the entire composition, or a weight ratio of low melt wax to high melt wax of greater than about 3:2, greater than about 2:1, greater than about 5:2, greater than about 3:1, or greater than about 4:1, can effectively impart stability to the composition.

In contrast, traditional use of low melt waxes is limited to very minor proportions, due to stability issues, and it is common in conventional practice, for example, to use a high loading of high melt waxes (or brittle waxes) to achieve stability at elevated temperatures. For example, low melt fatty acid esters have been provided in hollow particles in weight concentrations ranging from just 0.1 to 5%. See U.S. Pat. No. 5,914,117. It has been surprisingly found herein that low melt waxes can be heat-stabilized by combination of large relative amounts of the low melt waxes with small relative amounts of higher melt waxes.

For example, in certain preferred embodiments, the high melt wax comprises from about 5% to about 40% by weight, based on the total weight of the composition. In some embodiments, the high melt component comprises from at least about 10%, at least abut 15%, or at least about 20% by weight, based on the total weight of the composition. In other embodiments, the high melt component comprises no more than about 35%, no more than about 30%, no more than about 25%, or no more than about 20% by weight, based on the total weight of the composition. In other embodiments, the amount of high melt wax will be between about 2.5-5%, between about 5-10%, between about 10-15%, between about 15-20%, between about 20-25%, between about 25-30%, between about 30-35%, or between about 35-40% by weight of the total composition. Accordingly, some embodiments of the instant invention provide for lowering the load of high melt waxes in cosmetic compositions, e.g., in cosmetic sticks. The larger amounts of low melt material, and the corresponding smaller amounts of high melt wax(es), can impart better performance characteristics, e.g., with respect to feel and payoff, while maintaining stability.

In some embodiments, compositions comprising low melt wax(es) and a small proportion of high melt wax(es) can form stable solids, as well as stable semi-solids and/or gels. In certain embodiments, the compositions of the instant invention are not gels or are not gel-based, unlike, e.g., the gel-based compositions described in Avon Products's U.S. patent application Ser. No. 11/642,348, titled “High gloss gel-based lipstick,” and may be free of ester terminated poly(ester-amide) polymers, or comprise less than 0.1% by weight, less than about 0.08% by weight, less than about 0.05% by weight, less than about 0.02% by weight, or less than about 0.01% by weight of an ester terminated poly(ester-amide) polymer. In certain embodiments, compositions of the instant invention are not in the form of a powder and/or are not in the form of an emulsion. In non-emulsion forms, compositions described herein do not need an emulsifier, and are preferably free of emulsifier or substantially free of emulsifier, unlike the emulsions described in Intl Appl. Publ. No. WO 2006/007564, wherein a polymeric emulsifier must be used to emulsify, but not solubilize, an oil-soluble coolant that remains in its crystalline state. By substantially free of emulsifier is meant that the amount of emulsifier is so low as to not have a measurable impact on the stability of the composition, and in any event may be less than about 0.5% by weight, less than 0.1% by weight, less than 0.05% by weight, or less than 0.01% by weight of the composition.

In some embodiments, the low melt/high melt wax compositions are in the form of an emulsion. Unlike the compositions taught in WO 2006/007564, however, low melt wax/high melt wax compositions of the instant invention include at least one high melt wax, whereas the emulsions of WO 2006/007564 specifically exclude substances other than water-soluble materials. The high melt waxes described herein are preferably not water-soluble materials and are preferably not water-dispersible materials.

Some embodiments provide a composition consisting essentially of at least one low melt wax and at least one high melt wax. Unlike approaches excluding oil-soluble materials and relying on emulsions, the stability of the low melt wax/high melt wax compositions described herein allow the combination of different waxes to serve as the entire product, or nearly the entire product, save for minor amounts of customary ingredients such as preservatives, fragrances, colorants, and the like. As used in this context, “consisting essentially of” excludes other materials that measurably stabilize the low melt materials, such as polymeric emulsifiers and/or hollow particles, as well as other substances that would materially affect the ability of the high melt wax to stabilize the low melt material. For example, in some embodiments, the low melt and high melt wax components together collectively comprise from about 50% to as much as about 100% by weight of the total composition, preferably with the low melt wax homogenously dispersed with the high melt wax. In some embodiments, the low melt and high melt waxes together collectively comprise from about 30% to about 40%, from about 40% to about 50%, from about 50% to about 60%, from about 60% to about 70%, from about 70% to about 80%, from about 80% to about 90%, from about 90% to about 95%, or from about 95% to about 99% or as much as about 100% by weight, based on the weight of the total composition.

The remaining percent compositional components in such embodiments may comprise, e.g., pigments and other colorants, fragrances, preservatives, minor amounts (e.g., less than about 1% by weight) of biologically active components, and/or other additives customary for use in cosmetic compositions. This approach allows low melt waxes to be used in higher proportions in stable cosmetics, without including large amounts of high melt waxes or other substances traditionally needed to stabilize the low melt materials.

In certain particularly preferred embodiments, the composition comprises an amount of low melt wax(es) effective to provide a cooling sensation when applied to the skin. When a low melt wax, for example having melting point slightly less than that of human skin, is applied to the skin, the wax melts absorbing heat from the skin. The absorption of heat can translate to a cooling sensation on the external surface of the skin. Compositions that produce a cooling sensation upon contact with skin can be referred to as stimuli responsive compositions, in that they respond to certain stimuli (skin heat, rubbing, etc.) to produce a given effect. Compositions that produce a cooling sensation are also referred to herein as “cooling compositions” and the low melt material can be referred to as the “coolant.” Without wishing to be bound by theory, the effective amount of a low melt wax to produce a cooling sensation may depend on, for example, its enthalpy of fusion, melting temperature range, and/or purity, as described in more detail below.

In certain embodiments, the cooling composition comprises from about 30% to about 95% of low melt material by weight, and preferably from about 60% to about 95% of low melt material by weight, e.g., where the composition is in the form of a solid stick. In some embodiments, the cooling composition comprises from at least about 40%, at least about 50%, or at least about 60% by weight of the low melt material. In some embodiments, the cooling composition comprises as much as about 90%, at much as about 80%, or as much as about 70% by weight of the low melt material by weight. In some embodiments, a much higher percentage of low melt wax is used to achieve a cooling sensation, for example, as compared to usage levels in other approaches. In some embodiments, the low melt material will consist of, or consist essentially of one or more cooling waxes, by which is meant that the low melt material will comprise less than about 5% by weight, less than about 2.5% by weight, less than about 1% by weight, or less than about 0.5% by weight of other low melt components in addition to the cooling wax(es). In some embodiments, the at least one low melt cooling wax is present in an amount effective to provide a perceptible cooling sensation.

In certain embodiments, the low melt wax(es) is provided at a specified level of purity, for example, at a level of purity that allows a given amount of low melt wax to function effectively as a coolant. In some embodiments, the purity level of the low melt wax is from about 70% to about 100%. Typically, the purer the material, the greater its cooling effect because the melting range is broadened by the presence of impurities. In some embodiments, the low melt wax is at least about 75%, at least about 80%, or at least about 85% pure. In some embodiments, the low melt was is as much as about 85%, as much as about 90%, or as much as about 95% pure. In other embodiments, the low melt wax is 97% pure or greater, or even 99% by weight pure or greater. The purity of a low melt wax can be obtained by conventional methods known in the art, including chromatography or melting point determination using DSC.

In some embodiments, the composition of the invention will comprise, consist of, or consist essentially of only two waxes, meaning that the low melt wax component comprises a singular wax and the high melt wax component comprises a singular wax. In other words, compositions comprises three or more distinct waxes are excluded in some embodiments. In other embodiments, compositions comprises four or more distinct waxes are excluded.

Many suitable low melt waxes are emollients and therefore are useful as skin moisturizers. Particular mention may be made of fatty acid esters, including stearyl heptanoate. Accordingly, compositions according to the invention may be formulated as skin moisturizers and may include amounts of other ingredient customary in skin moisturizers, provided that the ingredients or the levels in which they are used do no adversely impair the stability of the composition. The compositions may also be formulated as skin cleansers, and in particular “water-free” skin cleansers due to the ability of various low melt materials to solubilize hydrophobic oils and debris on the skin. The products can be use to reduce “shine” on the skin caused by oil and sebum, and may be employed by rubbing a stick of the composition across the skin, such as the skin of the forehead, or may be used to remove makeup and the like from the skin.

In some embodiments, the composition further comprises at least one bioactive agent. “Bioactive agent” as used herein refers to any substance capable of having a desired biochemical and/or physiological effect on the human body, and can also be referred to as an “active ingredient.” Bioactive agents include, without limitation, compounds, including botanical extracts, that stimulate production of LOXL-1, inhibit calcineurin activity, and/or stimulate synthesis of GAGs. In one embodiment of the invention, the compositions may include additional bioactives including, but not limited to, botanicals, keratolytic agents, desquamating agents, keratinocyte proliferation enhancers, collagenase inhibitors, elastase inhibitors, depigmenting agents, anti-inflammatory agents, steroids, retinoids, anti-acne agents, antioxidants, alpha-hydroxy acids, including glycolic acid, beta-hydroxy acids, including salicylic acid or salicylates, thiodipropionic acid (TDPA) or esters thereof, ascorbic acid, advanced glycation end-product (AGE) inhibitors, and phytol and its derivatives.

Other bioactives that may be incorporated into compositions of the instant invention include, for example and without limitation, one or more hormonal compounds, alpha-keto acids, anti-aging agents, anti-wrinkle agents, anti-mycobacterial agents, antifungal agents, antiyeast agents, antibacterials, antimicrobials, antivirals, analgesics, lipidic compounds, anti-allergenic agents, H1 or H2 antihistamines, anti-inflammatory agents, corticosteroids, anti-asthmatic agents and bronchodilators, anti-irritants, antihyperkeratolytic agents, antineoplastics, immune system boosting agents, immune system suppressing agents, anesthetics, antiseptics, insect repellents, skin penetration enhancers, antipruritic agents, antiemetics, antimotion sickness agents, depigmenting agents, hypopigmenting agents, self-tanning agents, antiperspirants, antidermatitis agents, antipsoriatic agents, anti-acne agents, cardiovascular agents, vitamins, such as, e.g., tocopheryl acetate (vitamin E), vitamin A, beta carotene, salts and derivatives thereof, and other pharmaceutical agents or medicaments that can be applied topically to the skin and/or lips, as well as any combinations thereof.

When present, bioactive agents will comprises from about 0.0001% to about 20% by weight of the total weight of the composition. More typically, bioactive agents will comprises from about 0.001% to about 10% by weight, based on the total weight of the composition; and more typically from about 0.01% to about 5% by weight, and more typical still from about 0.1% to about 1% by weight based on the total weight of the composition. Preferably the bioactive is at a level that does not impair the stability of the composition.

The compositions described herein will find use as products for cooling, moisturizing, and/or cleansing skin, as well as for topically delivering bioactive agents in aesthetically pleasing vehicles. The composition may be formulated in a variety of product forms suitable for application to the skin, including without limitation, a pencil, a pomade, a pot, a stick, a nugget, or any other gel, solid, or semi-solid form. Preferred forms include solid self-supporting sticks.

The compositions can also include a thickener/gelling agent and/or a bulking agent that can act as structure-enhancing agents, although in some embodiments, gelling agents are excluded or are present at only very low levels, e.g., below about 0.1% by weight. For stick forms, the bulking agents may be present in an amount from about 1% to about 10% by weight of the stick. These agents provide body and strength to the formulations, for example, helping to make a stick free-standing and capable of retaining its original shape. Suitable bulking agents include, without limitation, powder fillers, mica, barium sulfate, nylon, talc, starch, calcium carbonate, silica, and mixtures thereof. Suitable structure-enhancing agents include clays, such as but not limited to, lithium magnesium silicate, also known as smectite clay, bentonite clay, also known as wilkinite, magnesium aluminum silicate, lithium magnesium silicate, montmorillonite represented by the formula Al₂O₃.4SiO₂.H₂O, and mixtures thereof. Lithium magnesium silicate and smectite clay are preferred.

In certain embodiments, the formulations involve emulsions. For example, the cosmetically acceptable vehicle used may comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like, for example, having the appearance of a cream, gel or micro-emulsions. The emulsion may include an emulsifier, such as a nonionic, anionic or amphoteric surfactant.

The aqueous phase of an emulsion can include water, a gelling agent, a humectant, a moisturizer, and the like. The water is preferably demineralized water. The aqueous phase of the emulsion may include one or more additional solvents, including lower alcohols, such as ethanol, isopropanol, and the like. The volatile solvent may also be a cosmetically and/or pharmaceutically acceptable ester such as butyl acetate or ethyl acetate; ketones such as acetone or ethyl methyl ketone; or the like. The gelling agent, can be, for example, one or more polymethacrylates, carbomers, cellulosics, water swellable Lucentite SWN, and Vee gums. A preferred gelling agent in some embodiments is glyceryl polymethacrylate. In some embodiments, a preferred humectant is glycerin. In some embodiments, a preferred moisturizer is hyaluronic acid sodium salt.

The oil phase of the emulsion may include, for example, vegetable oils; esters such as octyl palmitate, isopropyl myristate and isopropyl palmitate; ethers such as dicapryl ether; fatty alcohols such as cetyl alcohol, stearyl alcohol and behenyl alcohol; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; additional natural or synthetic waxes; and the like. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Non-limiting emulsifiers include emulsifying waxes, emulsifying polyhydric alcohols, polyether polyols, polyethers, mono- or di-ester of polyols, ethylene glycol mono-stearates, glycerin mono-stearates, glycerin di-stearates, silicone-containing emulsifiers, soya sterols, fatty alcohols such as cetyl alcohol, acrylates, fatty acids such as stearic acid, fatty acid salts, and mixtures thereof. The preferred emulsifiers include soya sterol, cetyl alcohol, stearic acid, emulsifying wax, acrylates, silicone containing emulsifiers and mixtures thereof. Other specific emulsifiers that can be used in the composition of the present invention include, but are not limited to, one or more of the following: Lauryl PEG-9 polydimethylsiloxane dimethicone; C₁₀₋₃₀ alkyl acrylate crosspolymer; Dimethicone PEG-7 isostearate, acrylamide copolymer; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene phenols such as polyoxyethylene octyl phenol and polyoxyethylene nonyl phenol; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11^{th} Edition 2006, the disclosure of which is hereby incorporated by reference.

These emulsifiers typically will be present in the composition in an amount from about 0.03% to about 10% by weight, in particular in an amount from about 0.3% to about 10% by weight, and more preferably from about 3% to about 5% by weight. An example of an emulsion stick formulation comprising 30-80% stearyl heptanoate, 10-20% high melt wax, and 3-5% emulsifier is provided below in Example 8. An example of an emulsion stick formulation comprising 30-80% octadecane, 10-20% high melt wax, and 3-5% emulsifier is provided below in Example 10. In some embodiments, the compositions are free of or substantially free of emulsifier.

The composition may comprise coloring agents, including pigments, pearlescent agents, lakes, and dyes, and the like. Coloring agents are well known in the art and are disclosed in the C.T.F.A. Cosmetic Ingredient Handbook, 12^{th} Edition, 2008, the contents of which are hereby incorporated by reference. Organic dyes include, for example, FD&C dyes, D&C dyes, including D&C Red, Nos. 2, 5, 6, 7, 10, 11, 12, 13, 30 and 34, D&C Yellow No. 5, Blue No. 1, and Violet No. 2. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, β-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides, as well as composite oxides and composite hydroxides, such as iron titanate, cobalt titanate and cobalt aluminate.

Other suitable colorants include ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, carbon black and the like. The colorants may be surface modified with, for example, fluoropolymers, to adjust one or more characteristics of the colorant as described in, for example, U.S. Pat. Nos. 6,471,950, 5,482,547, and 4,832,944, the contents of which are hereby incorporated by reference. Suitable pearling pigments include without limitation bismuth oxychloride, guanine and titanium composite materials containing, as a titanium component, titanium dioxide, titanium lower oxides or titanium oxynitride, as disclosed in U.S. Pat. No. 5,340,569, the contents of which are hereby incorporated by reference. The compositions may also include glittering agents. In the stick and/or nugget form, the color or pigment may be included in an amount from about 5% to about 20% by weight of the stick or nugget. An example of an color emulsion stick formulation comprising 30-60% stearyl heptanoate, 10-20% high melt wax, and 5-20% color or pigment is provided below in Example 9. An example of an color emulsion stick formulation comprising 30-60% octadecane, 10-20% high melt wax, and 5-20% color or pigment is provided below in Example 11.

The compositions may also include a fragrance, which may be included in an amount from about 0.001% to about 2.0% by weight of the composition. For example, a cooling lip balm or balm for sore muscles may be colored and/or scented to resemble the color and/or scent of mint; a product used as an under-eye treatment may be colored and/or scented to resemble the color and/or scent of cucumbers; a cleansing product may be colored and/or scented to resemble the color and/or scent of citrus, and the like.

The compositions of the present invention, and/or their vehicles if any, may contain other ingredients, such as, for example, one or more preservatives; one or more UV-absorbers (e.g. screening agents or sunscreens), stabilizers or antioxidants; lipid materials, and the like. Preservatives may be present in an amount from about 0.05% to about 2% of the composition. Preferred preservatives include: EDTA, phenoxyethanol, iodopropynylbutyl carbamate, 4-hydroxybenzoic acid, its esters and derivatives, such as methyl 4-hydroxybenzoate (methyl paraben), ethyl 4-hydroxybenzoate (ethyl paraben), propyl 4-hydroxybenzoate (propyl paraben), butyl 4-hydroxybenzoate (butyl paraben), and mixtures thereof. Particular mention may be made of caprylyl glycol as a preservative.

The compositions of the present invention may also contain UV-absorbers, UV stabilizers and antioxidants. The compositions of the present invention may also contain other cosmetic ingredients and excipients, including, but not limited to, fillers, film-formers, minerals, exfollients; other soothing and/or cooling agents, additional lubricants, additional moisturizers, additional stabilizers, proteins, fibers, photostabilizing agents, chelating agents, neutralizers (e.g., triethanolamine) and mixtures thereof. Compositions of the present invention may also include one or more additional emollients, e.g., to aid bioactive delivery, as described above. Some preferred emollients include, for example: polytriglyceryl erucate/eleostearate, avocado, lanolin, diisostearyl fumarate, myristyl lactate, and mixtures thereof.

The present invention provides methods for providing a cooling sensation to skin comprising applying to the skin a topical composition comprising at least one low melt wax in combination with a small proportion of at least one high melt wax, wherein the low melt wax(es) is at a purity level and/or in an amount effective to provide a cooling sensation perceptible to the user, and in particular a cooling sensation on the skin or lips. It is also believed that a cooling sensation caused by absorption of heat when the product is applied to the skin can have a longer and/or prolonged effect, e.g., compared to compositions relying on evaporants. Examples 1-6 below illustrate how low melt materials having relatively large enthalpies of fusion but relatively narrow melting temperature ranges produce a rapid drop in temperature that can be readily perceived as sensory cooling in preferred embodiments of the instant invention. The low melt waxes can be selected based on their melting temperature ranges, and/or enthalpies of fusion, to produce various desired cooling effects.

Cooling compositions described herein differ from many traditional products that produce sensory cooling by use of an evaporant. An “evaporant” as used herein refers to a substance used to produce a cooling sensation on skin based on the high volatility of the substance. That is, upon contact with skin, a volatile substance may evaporate off of the skin, thereby cooling the external skin temperature. For example, many cosmetics use evaporants such as ethanol or terpene alcohols, ammonium nitrate in water, and the like, to achieve a cooling effect. Additional evaporants are also disclosed, for example, in Jap. Pat. Appl. Pub. Nos. 08283305 and 59219208, the disclosures of which are hereby incorporated by reference. Another approach involves using menthol, that provides a cooling sensation by chemically triggering cold sensitive receptors in the skin, such as TRPM8 receptors. In some cases, these approaches have been associated with irritancy, stinging, odors, and/or a propensity to dry the skin.

In contrast, compositions of the instant invention produce sensory cooling based on absorbing heat from the skin (enthalpy of fusion); and/or by melting over a narrow range of temperatures (melting temperature range). In certain embodiments, the compositions of the invention exclude evaporants, enzymes, menthol, and/or other substances traditionally used to produce sensory cooling, and rather relies on the stabilized low melt wax(es) to produce cooling without irritation from evaporants, menthol, enzymes, and the like. In other embodiments, these conventional coolants can be used in addition to the cooling effect achieved by the low melt materials of the invention.

Some embodiments provide a method for treating lips. Lips tend to become dry and/or chapped, for example, as a result of diminished moisture in the skin on and around the lips. For example, dry lips may be due to low humidity, frequent licking of the lips, and/or an allergic reaction. Conditions associated with dry lips include the lips becoming chapped, cracked, blistered, uncomfortable, and painful. Such lip conditions can be treated, in some embodiments, by topically applying to an area of the lips suffering therefrom a composition of the instant invention, for example, a composition that provides a cooling sensation and/or a moisturizing benefit. The wax(es) of the composition can protect the lips, e.g., from adverse weather, including wind, rain or extended exposure to sunlight; while the cooling sensation can relieve and/or soothe pain or discomfort. The composition can take the form of a lip balm, lip gloss or lipstick, for example. The composition may also be useful for treating the area of skin under the eyes, which can become puffy, saggy, dry, and/or inflamed. Topical application of the compositions according to the invention to an under-eye area can provide a cooling sensation and can reduce puffiness, sagging, dryness and/or redness, soothe the tender skin, and/or produce a fresher, younger look around the eyes.

The composition are also contemplated to be useful for treating sunburned skin by topically applying to an area of sunburned skin a composition of the instant invention comprising an amount of low melt material effective to provide a cooling sensation to the skin to soothe or relive the discomfort or pain associated with the burn. For example, a cooling composition described herein can be used in the form of a solid stick as a balm for after-sun treatment and/or sunburn relief. Compositions according to this embodiment may comprise additional components for treating sunburn including, for example, aloe, lidocaine, benzocaine, vitamin E, hydrocortisone, and ascorbyl fatty acid ester (e.g., ascorbyl palmitate, ascorbyl laurate, ascorbyl myristate, ascorbyl stearate).

Some embodiments provide a method for treating muscle ache. Muscle ache, also called myalgia, refers to pain or discomfort experienced in a muscle or a group of muscles, sometimes accompanied by a feeling of malaise. The ache may be due to, for example, overuse and/or incorrect use of the muscles, and can be associated with sore, stiff, burning or knotted sensations, which can be sharp, chronic, or intermittent. Muscle ache can be treated, in some embodiments, by topically applying to an area suffering therefrom a composition of the instant invention, for example, a composition that provides a cooling sensation. The area suffering form muscle ache (and its associated conditions and/or symptoms) includes the area of skin over the aching muscle. The cooling sensation produced upon application of the composition to the skin over the muscles can, for example, soothe or relive the discomfort, pain and/or stiffness. For example, a cooling composition described herein can be used in the form of a solid stick as a muscle ache balm.

In addition, one of skill in the art will recognize other cosmetic, pharmaceutical, and personal care applications for the compositions described herein, and such applications are also contemplated as within the scope of the instant invention. For example, foot care products and underarm deodorants, e.g., an underarm stick that cools and/or soothes the skin under the arms, e.g., to provide relief after shaving. Compositions described herein can also find use in shaving creams and after-shave lotions.

### EXAMPLES

### Example 1

### Melting temperature range of Isostearyl Hydrostearate

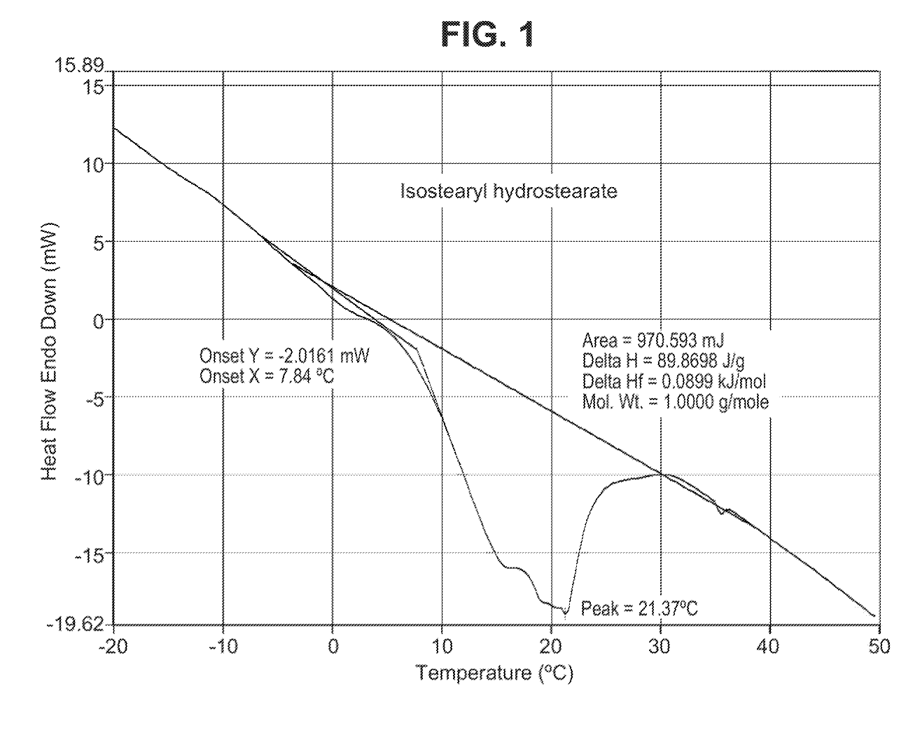

A DSC thermogram was obtained for isostearyl hydrostearate in accordance with the manufacturer's instructions, and is presented in FIG. 1. As FIG. 1 illustrates, isostearyl hydrostearate exhibits an onset temperature of 8° C. and a peak temperature of 21° C., giving a melting temperature range (difference between onset and peak) of 13° C. Its melting temperature range is thus over 12° C. Also, its enthalpy of fusion (ΔH) was determined to be 90 J/g. This material was found not to effectively function as a coolant in accordance with the instant invention although it is not excluded from the broader aspects of the invention where its delivery to the skin is desired.

### Example 2

### Melting Temperature Range of Glycerol Tridecanoate

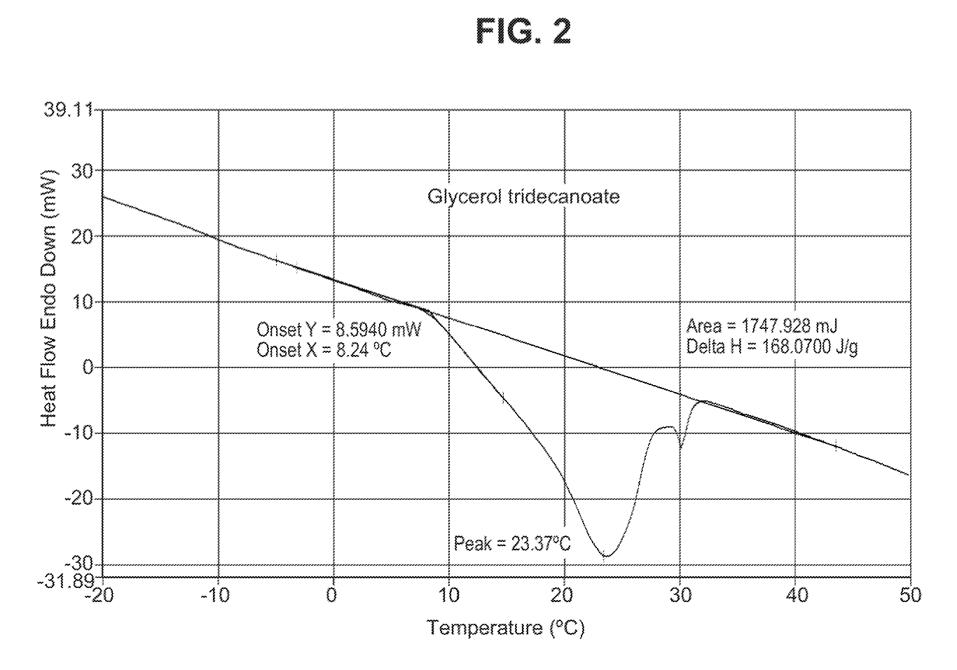

A DSC thermogram was obtained for glycerol tridecanoate, in accordance with the manufacturer's instructions, and is presented in FIG. 2 As FIG. 2 illustrates, glycerol tridecanoate exhibits an onset temperature of 8.6° C. and a peak temperature of 23.4° C., giving a melting temperature range (difference between onset and peak) of 14.8° C. The melting temperature range is thus over 12° C. Also, its enthalpy of fusion (ΔH) was found to be 167 J/g. This material also was found not to effectively function as a coolant, despite its relatively large enthalpy of fusion, although it is not excluded from the broader aspects of the invention where its delivery to the skin is desired.

### Example 3

### Melting Temperature Range of 89% Pure Stearyl Heptanoate

A DSC thermogram was obtained for stearyl heptanoate at 89% purity, in accordance with the manufacturer's instructions, and is presented in FIG. 3 As FIG. 3 illustrates, 89% pure stearyl heptanoate exhibits an onset temperature of 19.5° C. and a peak temperature of 26.4° C., giving a melting temperature range (difference between onset and peak) of 6.9° C. Notably, the melting temperature range in this case is under 12° C. Also, its enthalpy of fusion (ΔH) was found to be 149 J/g. 89% pure Stearyl Heptanoate was found to effectively function as a coolant in accordance with the instant invention, making it a preferred low melt wax in certain embodiments of the invention.

### Example 4

### Melting Temperature Range of 99% Pure Stearyl Heptanoate

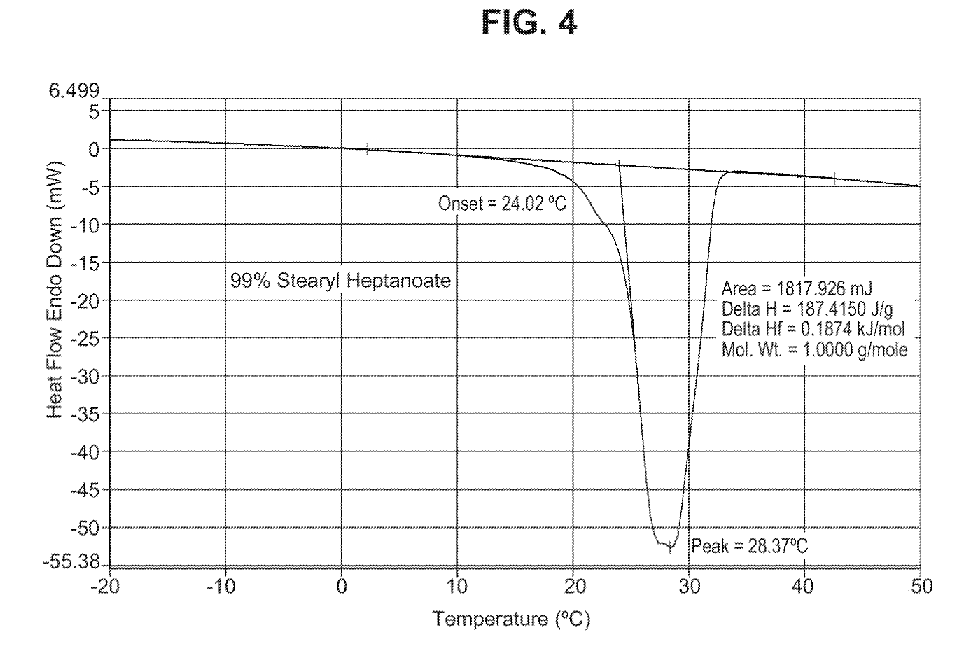

A DSC thermogram was obtained for stearyl heptanoate at 99% purity, in accordance with the manufacturer's instructions, and is presented in FIG. 4. As FIG. 4 illustrates, 99% pure stearyl heptanoate exhibits an onset temperature of 24.0° C. and a peak temperature of 28.4° C., giving a melting temperature range (difference between onset and peak) of 4.4° C. Notably, the melting temperature range in this case is again under 12° C. Also, its enthalpy of fusion (ΔH) was found to be 187 J/g. 99% pure Stearyl Heptanoate was found to effectively function as a coolant in accordance with the instant invention, making it a preferred low melt wax in certain embodiments.

### Example 5

### Melting Temperature Range of a 90% Stearyl Heptanoate Composition

A composition comprising 90% by weight stearyl heptanoate and 10% by weight of a 1:1 mixture of alkyl dimethicone (silicone wax) and paraffin wax was prepared, as illustrated in Table 3 below.

TABLE 3

| Components | Weight % |
|---|---|
| stearyl heptanoate | 90 |
| alkyl dimethicone (silicone wax) | 5 |
| paraffin wax | 5 |

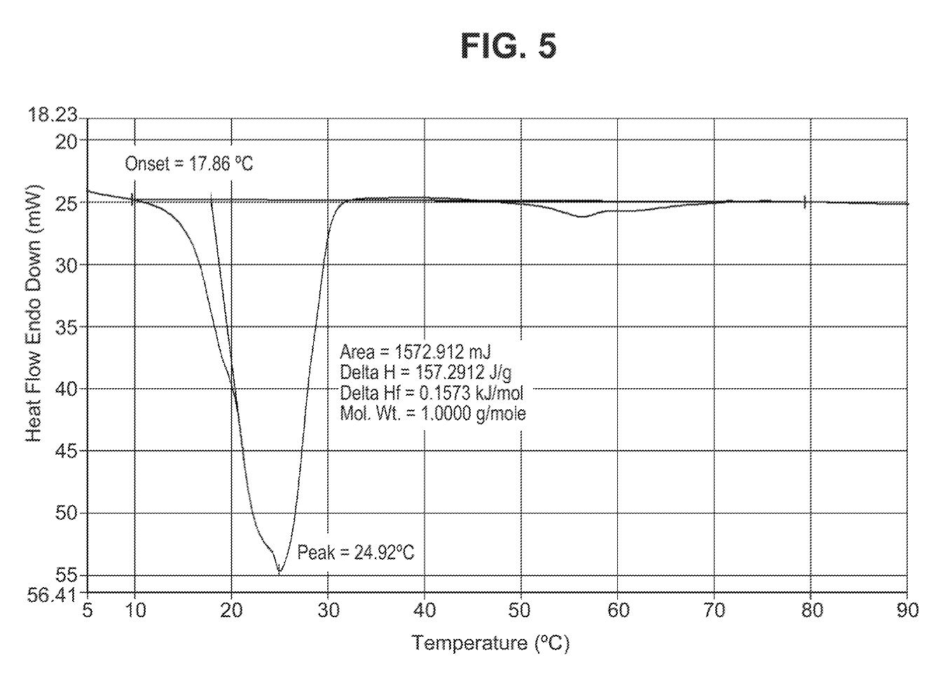

A DSC thermogram was obtained for this composition, in accordance with the manufacturer's instructions, and is presented in FIG. 5. As FIG. 5 illustrates, this composition comprising 90% stearyl heptanoate exhibits an onset temperature of 17.9° C. and a peak temperature of 24.9° C., giving a melting temperature range (difference between onset and peak) of 7.0° C. Notably, the melting temperature range in this case is again under 12° C. Also, its enthalpy of fusion (ΔH) was found to be 157 J/g. 90% stearyl heptanoate (a low melt wax) in a composition with 10% of high melt waxes was found to effectively function as a coolant in accordance with the instant invention, making such a composition preferred in certain embodiments where cooling is desired.

### Example 6

### Melting Temperature Range of an 80% Stearyl Heptanoate Composition

A composition was prepared comprising 80% by weight stearyl heptanoate and 20% by weight of a blend of different waxes in the following amounts: 12% paraffin wax, 4% ceresin wax, 2% candelilla wax, and 2% microcrystalline wax. The compositional components are illustrated in Table 4 below.

TABLE 4

| Components | Weight % |
|---|---|
| stearyl heptanoate | 80 |
| paraffin wax | 12 |
| ceresin wax | 4 |
| candelilla wax | 2 |
| microcrystalline wax | 2 |

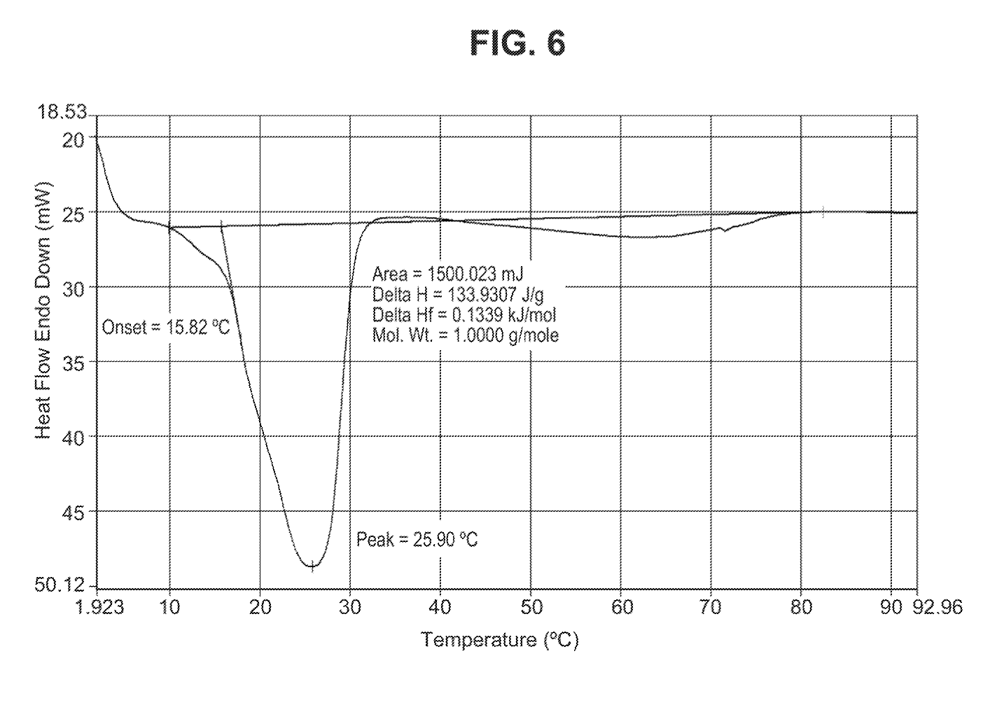

A DSC thermogram was obtained for this composition, in accordance with the manufacturer's instructions, and is presented in FIG. 6. As FIG. 6 illustrates, this composition comprising 80% stearyl heptanoate exhibits an onset temperature of 15.2° C. and a peak temperature of 25.9° C., giving a melting temperature range (difference between onset and peak) of 10.7° C. Notably, the melting temperature range in this case is again under 12° C. Also, its enthalpy of fusion (ΔH) was found to be 134 J/g. 80% stearyl heptanoate (a low melt wax) in a composition with 20% of high melt waxes was found to effectively function as a coolant in accordance with the instant invention, making such a composition also useful in certain embodiments.

### Example 7

### Anhydrous Stick Formulation Comprising Stearyl Heptanoate

Anhydrous stick formulations can be prepared by combining 80-90% of stearyl heptanoate (as the low melt wax) with 10-20% of high melt wax, e.g., as prepared in Examples 5 and 6 above, and illustrated in Tables 3 and 4. Such formulations can be used to provide a cooling sensation to skin, as well as to moisturize, cleanse the skin and remove makeup.

### Example 8

### Non-Color Emulsion Stick Formulation comprising Stearyl Heptanoate

An emulsion stick formulation can be prepared by combining 30-80% stearyl heptanoate (as the low melt wax) with 10-20% high melt wax. Additional components include: 3-5% emulsifier, 1-5% clay, 0.5-2% preservative, and water. The compositional components are illustrated in Table 5 below.

TABLE 5

| Components | Weight % |
|---|---|
| Stearyl heptanoate | 30-80 |
| High melt wax | 10-20 |
| emulsifier | 3-5 |
| clay | 1-5 |
| preservative | 0.5-2 |
| water | q.s |
| Total: | 100 |

### Example 9

### Color Emulsion Stick Formulation comprising Stearyl Heptanoate

A colored emulsion stick formulation can be prepared by combining 30-60% stearyl heptanoate (as the low melt wax) with 10-20% high melt wax. Additional components include 5-10% fillers, 1-5% film former, 5-20% color or pigment, 3-5% emulsifier, 0.5-2% preservative, and water. The compositional components are illustrated in Table 6 below.

TABLE 6

| Components | Weight % |
|---|---|
| Stearyl heptanoate | 30-60 |
| High melt wax | 10-20 |
| fillers | 5-10 |
| film former | 1-5 |
| color/pigment | 5-20 |
| emulsifier | 3-5 |
| preservative | 0.5-2 |
| water | q.s |
| Total: | 100 |

### Example 10

### Non-Color Emulsion Stick Formulation Comprising Octadecane

An emulsion stick formulation can be prepared by combining 30-80% octadecane (as the low melt wax) with 10-20% high melt wax. Additional components include: 3-5% emulsifier, 1-5% clay, 0.5-2% preservative, and water. The compositional components are illustrated in table 7 below.

TABLE 7

| Components | Weight % |
|---|---|
| Octadecane | 30-80 |
| High melt wax | 10-20 |
| emulsifier | 3-5 |
| clay | 1-5 |
| preservative | 0.5-2 |
| water | q.s |
| Total: | 100 |

### Example 11

### Color Emulsion Stick Formulation comprising Octadecane

A colored emulsion stick formulation can be prepared by combining 30-60% Octadecane (as the low melt wax) with 10-20% high melt wax. Additional components include 5-10% fillers, 1-5% film former, 5-20% color or pigment, 3-5% emulsifier, 0.5-2% preservative, and water. The compositional components are illustrated in Table 8 below.

TABLE 8

| Components | Weight % |
|---|---|
| Octadecane | 30-60 |
| High melt wax | 10-20 |
| fillers | 5-10 |
| film former | 1-5 |
| color/pigment | 5-20 |
| emulsifier | 3-5 |
| preservative | 0.5-2 |
| water | q.s |
| Total: | 100 |

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A composition comprising (i) at least 25% by weight of a first wax component capable of melting on contact with human skin and (ii) a second wax component having a melting point greater than about 50° C. in an amount sufficient to stabilize said first wax such that said composition exists as a self-supporting solid or semi-solid at 30° C.

2. The composition according to claim 1, wherein said first wax component is characterized by a difference between onset and peak melting temperatures of less than about 12° C. such that a perceptible cooling sensation is detected when said first wax component melts on contact with human skin.

3. The composition according to claim 2, wherein said difference between onset and peak melting temperatures is less than about 7° C.

4. The composition according to claim 1, wherein said first wax component comprises stearyl heptanoate.

5. The composition according to claim 1, wherein said first wax component comprises octadecane.

6. The composition according to claim 1, wherein said second wax component has a melting point greater than about 60° C.

7. The composition according to claim 1, wherein said second wax component has a melting point greater than about 70° C.

8. The composition according to claim 1, wherein said second wax component has a melting point greater than about 80° C.

9. The composition according to claim 1, wherein said second wax component comprises paraffin wax.

10. The composition according to claim 1, wherein said high melt wax comprises from about 5% to about 40% of said composition.

11. The composition according to claim 1, wherein said composition excludes a silicone fluid or volatile silicone; and wherein said low melt material is not encapsulated.

12. The composition according to claim 11, wherein said composition is in the form of form of a self-supporting stick.

13. The composition according to claim 1, wherein said composition is not an emulsion.

14. The composition according to claim 1, wherein said composition is an emulsion.

15. A composition comprising (i) at least 25% by weight of octadecane and (ii) a high melting wax component having a melting point greater than about 50° C. in an amount sufficient to stabilize said first wax such that said composition exists as a self-supporting solid or semi-solid at 30° C., said composition being capable of providing a cooling sensation when topically applied to the skin.

16. The composition according to claim 15, wherein said high melting wax component has a melting point greater than about 70° C.

17. The composition according to claim 15, wherein said high melting wax component comprises paraffin wax.

18. The composition according to claim 15, wherein said high melting wax component comprises from about 5% to about 40% of said composition.

19. A method for providing a cooling sensation to the skin comprising topically applying to the skin the compositions according, to claim 1 or 15.

20. A lip product in the form of a stick comprising the compositions according to claim 1 or 15.
